(19) United States
(12) Patent Application Publication
(10) Pub. No.: US 2019/0176768 A1
(43) Pub. Date: Jun. 13, 2019

(54) GERM-FREE RIDE SYSTEM FOR AUTONOMOUS SHARED FLEETS

(71) Applicant: GM Global Technology Operations LLC, Detroit, MI (US)

(72) Inventors: Jose Ezequiel DIAZ GARCIA, Metepec (MX); Alejandro HERNANDEZ COVARRUBIAS, Metepec (MX); Armando VERA GARCIA, Toluca (MX)

(73) Assignee: GM Global Technology Operations LLC, Detroit, MI (US)

(21) Appl. No.: 15/838,625

(22) Filed: Dec. 12, 2017

(51) Int. Cl. B60S 1/64; B60H 3/00; A61L 9/14; A61L 2/22

(52) U.S. Cl. CPC B60S 1/64; B60H 3/0035; B60H 3/0014; A61L 9/14; A61L 2209/13; A61L 2202/14; A61L 2202/15; A61L 2202/25; A61L 2209/111; A61L 2/22

(57) ABSTRACT

A system includes a reservoir located in a vehicle. The reservoir is configured to store a substance for eliminating at least one of germs and odors from an interior of the vehicle. The system further includes a dispenser fluidly coupled to the reservoir. The dispenser is configured to dispense the substance into the interior of the vehicle. The system further includes a pump configured to pump the substance from the reservoir to the dispenser. The system further includes a controller configured to detect when an occupant of the vehicle exits the vehicle. In response to detecting the occupant of the vehicle exiting the vehicle, the controller is further configured to activate the pump to dispense the substance from the dispenser into the interior of the vehicle to eliminate at least one of the germs and the odors from the interior of the vehicle.

### INTRODUCTION

The information provided in this section is for the purpose of generally presenting the context of the disclosure. Work of the presently named inventors, to the extent it is described in this section, as well as aspects of the description that may not otherwise qualify as prior art at the time of filing, are neither expressly nor impliedly admitted as prior art against the present disclosure.

The present disclosure relates generally to systems and methods for disinfecting and refreshing the interior environment of vehicles and more particularly to a germ-free ride system for autonomous shared fleets.

Vehicles including taxis, rental cars, and fleets operated by businesses are typically shared by many people. The interior of these vehicles often needs to be cleaned to remove objects such as trash that are discarded by occupants. For example, the vehicles may need to be vacuumed and hand-cleaned to remove trash after each use. The interior also needs to be cleaned after each use to provide a feeling of freshness for the next occupant(s). For example, air fresheners may be installed and periodically replaced in these vehicles. Aerosols may also be sprayed after each use to provide a fresh smell for the next occupant(s).

### SUMMARY

A system comprises a reservoir located in a vehicle. The reservoir is configured to store a substance for eliminating at least one of germs and odors from an interior of the vehicle. The system further comprises a dispenser fluidly coupled to the reservoir. The dispenser is configured to dispense the substance into the interior of the vehicle. The system further comprises a pump configured to pump the substance from the reservoir to the dispenser. The system further comprises a controller configured to detect when an occupant of the vehicle exits the vehicle. In response to detecting the occupant of the vehicle exiting the vehicle, the controller is further configured to activate the pump to dispense the substance from the dispenser into the interior of the vehicle to eliminate at least one of the germs and the odors from the interior of the vehicle.

In other features, the controller is configured to detect when the occupant of the vehicle exits the vehicle based on an operation of a door of the vehicle. Alternatively or additionally, the controller is configured to detect when the occupant of the vehicle exits the vehicle based on sensing a change of weight on a seat of the vehicle.

In other features, the controller is configured to activate the pump after a predetermined amount of time has elapsed subsequent to the occupant exiting the vehicle. Alternatively or additionally, the controller is configured to activate the pump after a predetermined amount of time has elapsed subsequent to the vehicle being turned off.

In other features, the reservoir and the pump are integrated as a single unit that is connected to the dispenser by a conduit. Alternatively or additionally, the reservoir, the pump, and the dispenser are integrated as a single unit.

In other features, the system further comprises conduits configured to connect the reservoir, the pump, and the dispenser.

In other features, the system further comprises a sensor configured to sense a level of the substance in the reservoir and an indicator configured to indicate the level of the substance in the reservoir.

In other features, the system further comprises a sensor configured to sense a level of the substance in the reservoir, and the controller is configured to provide an indication of the level of the substance in the reservoir sensed by the sensor.

In other features, at least one of the reservoir and the pump communicates wirelessly with the controller.

In other features, the reservoir and the pump are integrated as a single unit that is connected to the dispenser by a conduit and that communicates wirelessly with the controller.

In other features, the reservoir, the pump, and the dispenser are integrated as a single unit that communicates wirelessly with the controller.

In still other features, a method comprises storing, in a reservoir located in a vehicle, a substance for eliminating at least one of germs and odors from an interior of the vehicle. The method further comprises detecting when an occupant of the vehicle exits the vehicle. In response to detecting the occupant of the vehicle exiting the vehicle, the method further comprises activating a pump to dispense the substance from a dispenser into the interior of the vehicle, pumping the substance from the reservoir to the dispenser, and dispensing the substance from the dispenser into the interior of the vehicle to eliminate at least one of the germs and the odors from the interior of the vehicle.

In other features, the method further comprises detecting when the occupant of the vehicle exits the vehicle based on an operation of a door of the vehicle. Alternatively or additionally, the method further comprises detecting when the occupant of the vehicle exits the vehicle based on sensing a change of weight on a seat of the vehicle.

In other features, the method further comprises activating the pump after a predetermined amount of time has elapsed subsequent to the occupant exiting the vehicle. Alternatively or additionally, the method further comprises activating the pump after a predetermined amount of time has elapsed subsequent to the vehicle being turned off.

In other features, the method further comprises sensing a level of the substance in the reservoir, and indicating the level of the substance in the reservoir.

In other features, the method further comprises wirelessly communicating with at least the pump.

Further areas of applicability of the present disclosure will become apparent from the detailed description, the claims and the drawings. The detailed description and specific examples are intended for purposes of illustration only and are not intended to limit the scope of the disclosure.

### BRIEF DESCRIPTION OF THE DRAWINGS

The present disclosure will become more fully understood from the detailed description and the accompanying drawings, wherein:

FIG. 1 shows an example of a vehicle including a system for disinfecting and refreshing an interior of the vehicle according to the present disclosure;

FIG. 2 shows an example of a reservoir and pump assembly used in the system for disinfecting and refreshing an interior of the vehicle according to the present disclosure;

FIG. 3 shows an example of a location where the of the reservoir and pump assembly of FIG. 2 can be installed in the vehicle according to the present disclosure;

FIG. 4 shows an example of a nozzle assembly used in the system for disinfecting and refreshing an interior of the vehicle according to the present disclosure;

FIG. 5 shows a nozzle of the nozzle assembly of FIG. 4;

FIG. 6 shows an example of a nozzle assembly used in the system for disinfecting and refreshing an interior of the vehicle according to the present disclosure;

FIG. 7 shows a functional block diagram of the system for disinfecting and refreshing an interior of the vehicle according to the present disclosure;

FIG. 8 shows a flowchart of a method for disinfecting and refreshing an interior of the vehicle according to the present disclosure; and

FIG. 9 shows a flowchart of the method of FIG. 8 is further detail.

In the drawings, reference numbers may be reused to identify similar and/or identical elements.

### DETAILED DESCRIPTION

Autonomous vehicles are on the horizon. Such vehicles may transport a person P1 from point A to point B, a person P2 from point B to point C, and so on. There may be no opportunity for someone to clean and refresh the interior of the vehicle after P1 leaves the vehicle and before P2 begins using the vehicle. P1 may leave behind odors, germs, and so on in the vehicle that may affect P2.

The present disclosure relates generally to systems and methods for automatically disinfecting and refreshing the interior environment of vehicles and more particularly to a germ-free ride system for autonomous shared fleets. The term automatically here means that the cleaning task is not performed by a person. Rather, the cleaning task is performed using an electronically controlled dispensing system installed in selected areas within the vehicle as explained below in detail.

The systems and methods of the present disclosure address potential negative perceptions consumers may have about shared vehicles that shared vehicles are dirty and unhygienic, have bad odors, etc. The systems and methods reduce the downtime of autonomous shared vehicles resulting from conducting deep cleansing procedures traditionally performed after each use. The systems and methods eliminate viruses, spores, bacteria, fungi, etc. from the interior of shared and autonomous vehicles. The systems and methods are automatically activated upon passenger(s) exiting the vehicle to ensure a germ-free, clean, and hygienic ride for the next passenger(s). The systems and methods use advanced products (e.g., vaporizing agents) that eliminate almost all viruses, spores, bacteria, fungi, etc. in the vehicle. The systems and methods can also be used to add aroma to confined spaces such as vehicle interior.

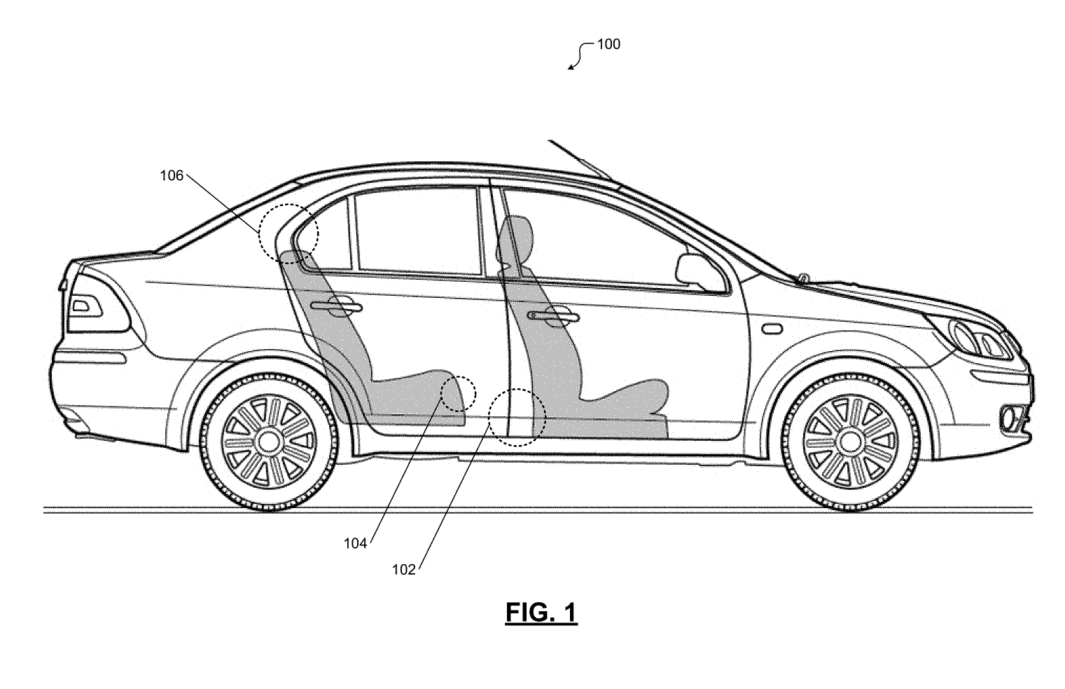

The present disclosure is organized as follows. Examples of implementing the systems and methods according to the present disclosure are initially presented with reference to FIGS. 1-6. Subsequently, the systems and methods are described in detail with reference to FIGS. 7-9.

FIGS. 1-6 show many ways of integrating the systems and methods according to the present disclosure into the vehicle interior. For example, the systems and methods can be integrated into the vehicle interior in the form of a germ-killer/cleanser reservoir with pump and one or more nozzles/vaporizers. The reservoir with pump and the nozzles/vaporizers can be installed at various locations (e.g., floor trim, rear window trim, etc.) in the vehicle as shown in FIGS. 1-6 and as explained below.

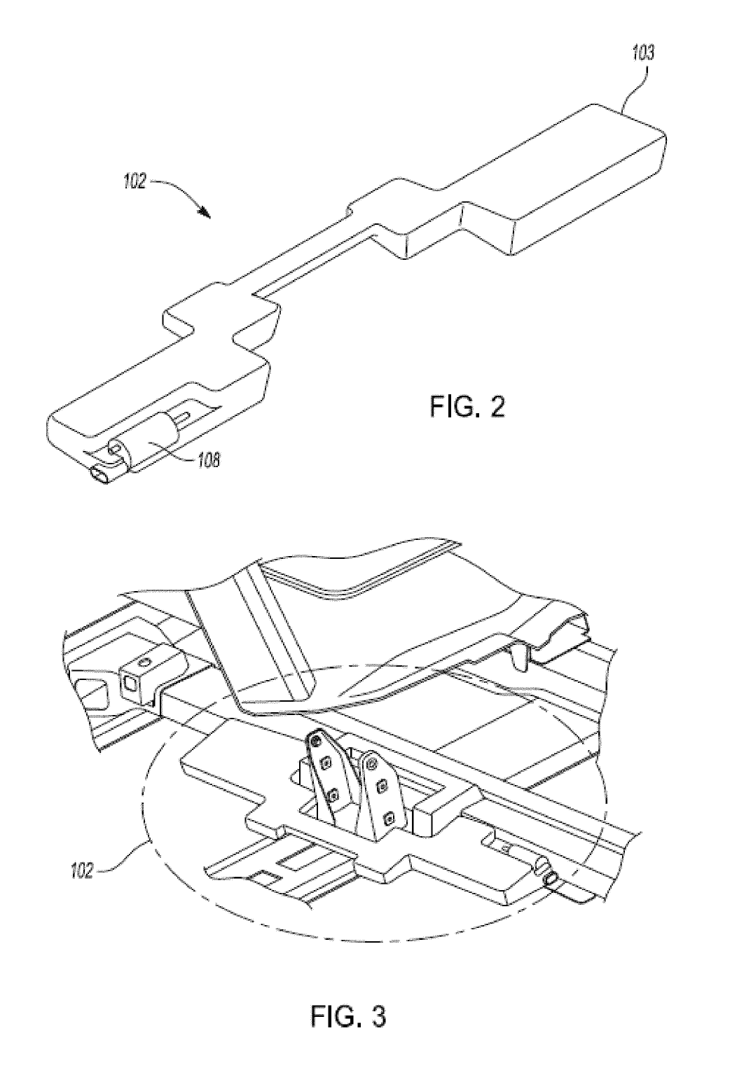

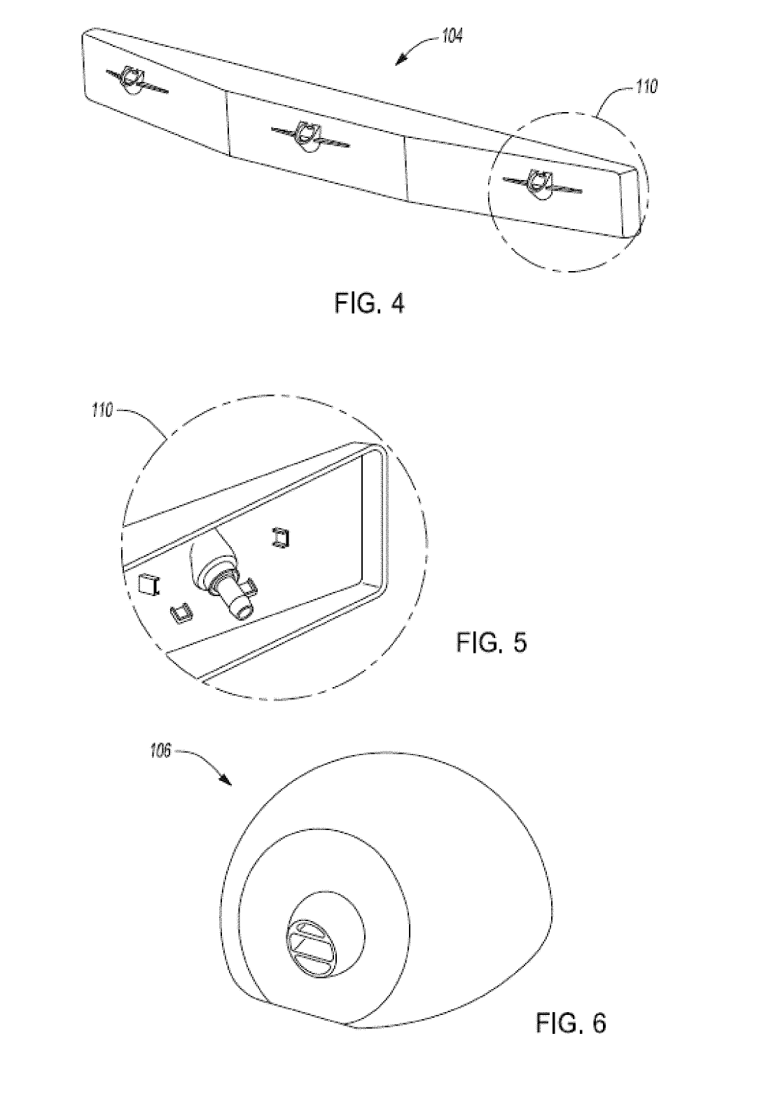

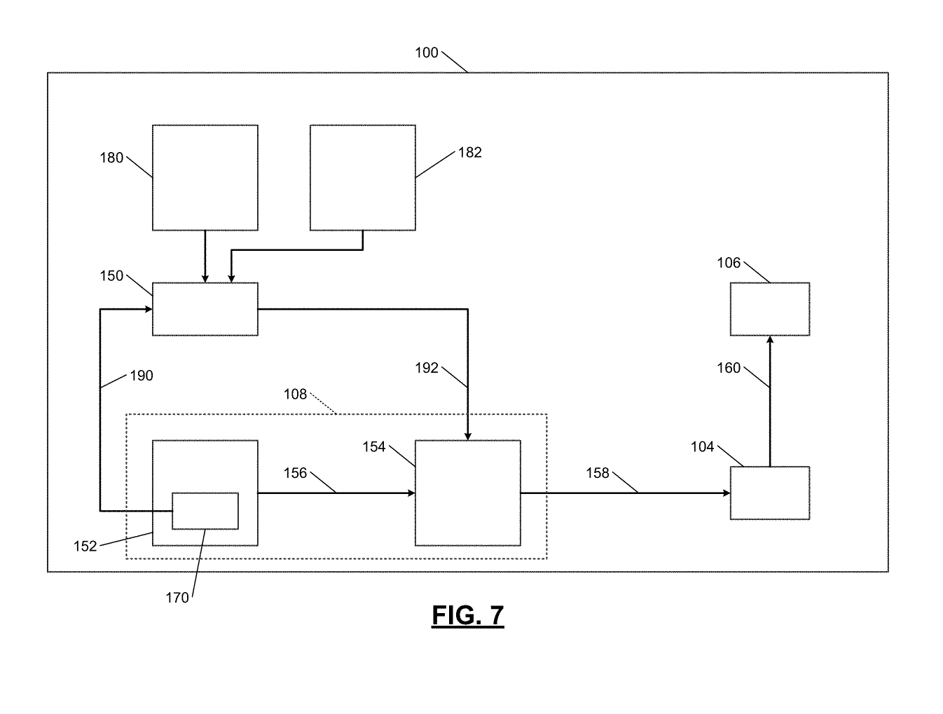

FIG. 1 shows a vehicle 100 including a reservoir and pump assembly 102, a nozzle assembly 104, and a nozzle assembly 106. The reservoir and pump assembly 102 supplies a germ-killer/cleanser agent to the nozzle assembly 104 and the nozzle assembly 106. The nozzle assembly 104 and the nozzle assembly 106 (each also called a dispenser) dispense the germ-killer/cleanser agent into different areas of the interior of the vehicle 100. An example of the reservoir and pump assembly 102 is shown in FIGS. 2-3. An example of the nozzle assembly 104 is shown in FIGS. 4-5. An example of the nozzle assembly 106 is shown in FIG. 6.

FIG. 2 shows an example of the reservoir and pump assembly 102. For example, the reservoir and pump assembly 102 can include a reservoir with an integrated pump (hereinafter a reservoir pump assembly) 108. For example, the reservoir and pump assembly 102 can include a blow-molded structure 103. The reservoir pump assembly 108 can be mounted in the blow-molded structure 103. The reservoir of the reservoir pump assembly 108 can store the germ-killer/cleanser agent. The pump of the reservoir pump assembly 108 can be activated as described below to pump the germ-killer/cleanser agent from the reservoir to one or more of the nozzle assembly 104 and the nozzle assembly 106. The nozzle assembly 104 and the nozzle assembly 106 dispense the germ-killer/cleanser agent into different areas of the interior of the vehicle 100.

FIG. 3 shows an example of a location where the of the reservoir and pump assembly 102 can be installed in the vehicle 100. For example, the reservoir and pump assembly 102 can be installed in the vehicle 100 by replacing existing floor trim expanded polypropylene (EPP) fillers. The reservoir and pump assembly 102 can be installed at other suitable locations in the vehicle 100.

FIG. 4 shows an example of the nozzle assembly 104. For example, the nozzle assembly 104 can include one or more nozzles 110. FIG. 5 shows an example of one of the nozzles 110. For example, the nozzle assembly 104 can be integrated into the floor trim of the vehicle 100 under a passenger seat as shown in FIG. 1. For example, the nozzle assembly 104 can dispense the germ-killer/cleanser agent into the lower cabin of the vehicle 100. The nozzle assembly 104 can be installed at other suitable locations in the vehicle 100. Additional nozzle assemblies 104 can be installed at other suitable locations in the vehicle 100. The nozzle assembly 104 can dispense the germ-killer/cleanser agent received from the reservoir and pump assembly 102. In some implementations, the nozzle assembly 104 can be equipped with its own reservoir pump assembly 108 and can be controlled and operated as a standalone integrated unit as described below.

FIG. 6 shows an example of the nozzle assembly 106. For example, the nozzle assembly 106 can include a single nozzle and can be integrated into the rear window trim of the vehicle 100 as shown in FIG. 1. For example, the nozzle assembly 106 can dispense the germ-killer/cleanser agent into the upper cabin of the vehicle 100. The nozzle assembly 106 can be installed at other suitable locations in the vehicle 100. Additional nozzle assemblies 106 can be installed at other suitable locations in the vehicle 100. The nozzle assembly 106 can dispense the germ-killer/cleanser agent received from the reservoir and pump assembly 102. In some implementations, the nozzle assembly 106 can be equipped with its own reservoir pump assembly 108 and can be controlled and operated as a standalone integrated unit as described below.

FIG. 7 shows an example of a controller 150 that controls the reservoir and pump assembly 102, the nozzle assembly 104 (shown as a dispenser), and the nozzle assembly 106 (shown as a dispenser). The reservoir and pump assembly 102 is shown to include a reservoir 152 and a pump 154. In some implementations, the reservoir 152 and the pump 154 may not be integrated. The reservoir 152 is connected to the pump 154 by a conduit 156. The reservoir and pump assembly 102 is connected to the nozzle assembly 104 by a conduit 158. The nozzle assembly 104 is connected to the nozzle assembly 106 by a conduit 160.

In some implementations, the reservoir and pump assembly 102 may be connected directly to the nozzle assembly 104 and directly to the nozzle assembly 106 by separate, respective conduits. In other words, both daisy chained connections (as shown) and star connections (as described) of the dispensers to the reservoir and pump assembly 102 are contemplated. In implementations where the reservoir 152 and the pump 154 are not integrated, the reservoir 152 is connected to the pump 154 by the conduit 156, and the pump 154 is connected directly to the nozzle assembly 104 and directly to the nozzle assembly 106 by separate, respective conduits to form a star connection.

While two dispensers are shown, the vehicle 100 can include one or more dispensers (i.e., a single dispenser or a plurality of dispensers). Further, the one or more dispensers may include multiple nozzles (e.g., as in the nozzle assembly 104) and/or a single nozzle (e.g., as in the nozzle assembly 106). In other words, the vehicle may include one or more of the nozzle assembly 104 and/or one or more of the nozzle assembly 106. Furthermore, while the dispensers are shown connected to the reservoir and pump assembly 102 and/or to another dispenser, as mentioned above, each dispenser can be equipped with its own reservoir pump assembly 108 and can be controlled and operated as a standalone integrated unit.

The controller 150 can sense an occupant entering and/or exiting the vehicle 100 using one or more occupant sensors 180 and can control the dispensing of the germ-killer/cleanser agent into the interior of the vehicle 100 based on the sensing (e.g., after the occupant exits the vehicle 100). For example, the occupant sensors 180 may include a sensor that senses when a door of the vehicle 100 opens. The opening of the door can indicate an occupant entering or exiting the vehicle 100. For example, the opening of the door coupled with turning the vehicle 100 on can indicate an occupant entering the vehicle 100, and the opening of the door coupled with turning the vehicle 100 off can indicate an occupant exiting the vehicle 100.

Further, the controller 150 may confirm that an occupant has permanently exited the vehicle 100 after waiting for a period of time after the opening of the door is sensed. For example, the controller 150 may conclude that an occupant has permanently exited the vehicle 100 when a predetermined time has elapsed after the opening of the door is sensed. The controller 150 may also conclude that an occupant has permanently exited the vehicle 100 when a predetermined time has elapsed after the vehicle 100 is turned off. This can be helpful in preventing dispensing of the germ-killer/cleanser agent when the occupant temporarily exits the vehicle 100 (e.g., to purchase something at a store, to use rest area, and so on) and returns to the vehicle 100 before a predetermined time has elapsed after the opening of the door is sensed (or after the vehicle 100 is turned off).

As another example, the occupant sensors 180 can include a sensor in a seat of the vehicle 100 that senses a presence or absence of a weight on the seat and that detects a presence or absence of an occupant in the seat based on sensing the weight. As yet another example, a camera in the vehicle 100 can monitor entry and exit of an occupant into and from the vehicle 100. Other ways of sensing entry and exit of an occupant into and from the vehicle 100 are contemplated.

In some implementations, an occupant may manually activate dispensing of the germ-killer/cleanser agent into the interior of the vehicle 100 using a manual activation switch 182. The occupant may activate the manual activation switch 182 while exiting the vehicle 100 (or upon entering into the vehicle 100). The controller 150 can control the dispensing of the germ-killer/cleanser agent into the interior of the vehicle 100 based on the occupant activating the manual activation switch 182.

Based on the sensing or the manual activation described above, the controller 150 activates the dispensing of the germ-killer/cleanser agent into the interior of the vehicle 100. Specifically, the controller 150 activates the pump 154 after determining that the occupant has exited the vehicle 100. The pump 154 pumps the germ-killer/cleanser agent from the reservoir 152 into the nozzle assembly 104 and the nozzle assembly 106. The nozzle assembly 104 and the nozzle assembly 106 dispense the germ-killer/cleanser agent. The germ-killer/cleanser agent vaporizes on dispensing and eliminates germs and odors in the dispensed areas.

The reservoir 152 includes a level sensor 170 to sense a level of the germ-killer/cleanser agent in the reservoir 152. The controller 150 monitors the level of the germ-killer/cleanser agent in the reservoir 152 based on the level sensed by the level sensor 170. The controller 150 can display the level of the germ-killer/cleanser agent on the dashboard of the vehicle 100, sound an alarm in the vehicle 100, or send the level information to a remote device such as a smartphone to trigger refilling of the germ-killer/cleanser agent into the reservoir 152.

In some implementations, the level of the germ-killer/cleanser agent may be indicated on the reservoir 152 using a suitable indicator. For example, an LED may blink or be lit on the reservoir 152 to indicate a low level of the germ-killer/cleanser agent in the reservoir 152 and to prompt a refill of the germ-killer/cleanser agent in the reservoir 152. In another example, a display on the reservoir 152 may indicate the level of the germ-killer/cleanser agent in the reservoir 152, and so on.

In some implementations, the connections between the controller 150 and the other elements shown in FIG. 7 may be wireless connections. For example, the connections 190 and 192 of the controller 150 to the level sensor 170 and the pump 154 may be wireless connections. The connections of the controller 150 to one or more of the occupant sensors 180 and the manual activation switch 182 may also be wireless connections. Further, if each dispenser is equipped a respective reservoir pump assembly 108 and operates as standalone integrated unit, the connection between the controller 150 and each dispenser can also be a wireless connection.

The wireless connections mentioned above can be Bluetooth and/or WiFi connections. If WiFi connections are used, the controller 150 may operate as an access point and all other elements may operate as client stations in an infrastructure mode compliant with one or more IEEE 802.1x specifications. Alternatively, the controller 150 and all other elements may operate as client stations in an ad-hoc mode compliant with one or more IEEE 802.1x specifications.

In some implementations, the controller 150 may be implemented as a module. In some implementations, the reservoir pump assembly 108 may include a module that operates the pump 154 according to control signals (i.e., data or instructions) received from the controller 150. Further, in some implementations, when the dispensers are equipped with respective reservoir pump assemblies 108 and operate as standalone integrated units, the dispensers may also include modules that operate the respective pumps 154 according to the control signals (i.e., data or instructions) received from the controller 150.

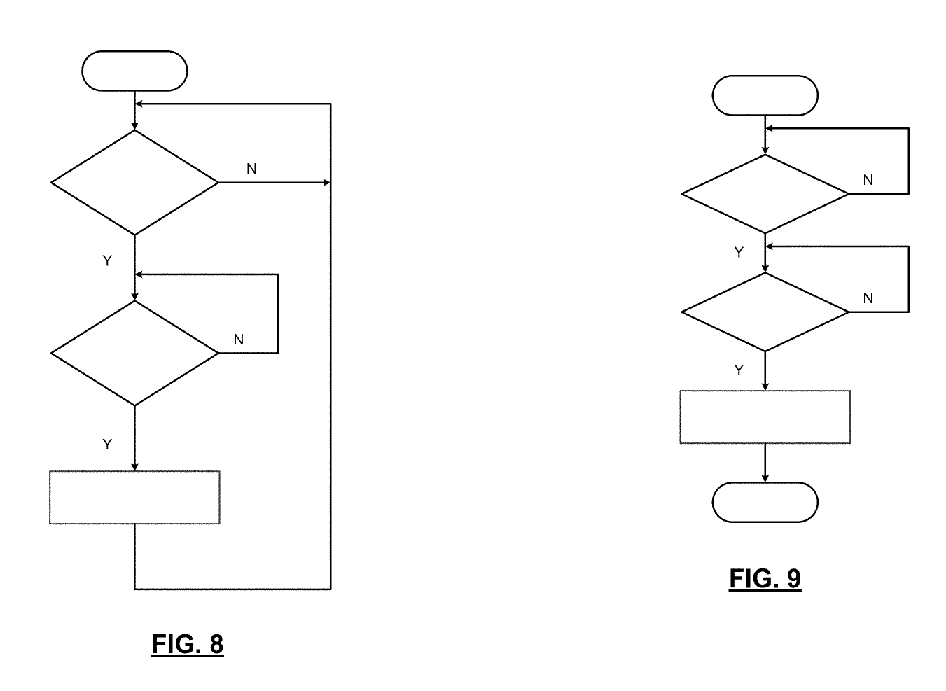

FIGS. 8 and 9 show methods for disinfecting and refreshing the interior environment of vehicles according to the present disclosure. FIG. 8 shows a method 200 for disinfecting and refreshing the interior environment of vehicles (e.g., using the system shown in FIGS. 1-7). FIG. 9 shows additional details of the method 200. In FIGS. 8 and 9, the term control indicates operations performed by the controller 150 shown in FIG. 7.

In FIG. 8, at 202, control determines whether the vehicle 100 is occupied. Control waits if the vehicle 100 is unoccupied. If the vehicle is occupied, at 204, control determines if the occupant or occupants have exited the vehicle 100. Control waits until the occupant or occupants have exited the vehicle 100. If the occupant or occupants have exited the vehicle 100, at 206, control activates the disinfecting and deodorizing system (e.g., the pump 154 and the dispensers) to disinfect and deodorize the vehicle 100.

In FIG. 9, a method 250 for confirming that the occupant or occupants have exited the vehicle 100 and then activating the disinfecting and deodorizing system to disinfect and deodorize the vehicle 100 is shown. At 252, control determines whether the occupant or occupants have exited the vehicle 100. Control waits until the occupant or occupants have exited the vehicle 100. If the occupant or occupants have exited the vehicle 100, at 254, control determines whether a predetermined amount of time has elapsed after the occupant or occupants have exited the vehicle 100 (e.g., after sensing opening of a door and optionally turning off the vehicle 100). Control waits until the predetermined amount of time has elapsed after the occupant or occupants have exited the vehicle 100. If the predetermined amount of time has elapsed after the occupant or occupants have exited the vehicle 100, at 256, control operates the pump 154 to spray the germ-killer/cleanser agent through the dispensers in the vehicle 100.

The utility of the systems and methods of the present disclosure is not limited to vehicles. The systems and methods can be used in any confined spaces that are used by different people at different times. For example, the systems and methods can also be used in ships, aircrafts, and so on. Further, the systems and methods can also be used in public places such as hotels, restaurants, libraries, office spaces (e.g., conference rooms), and also in homes. These examples are non-limiting, and other uses are contemplated.

The foregoing description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. It should be understood that one or more steps within a method may be executed in different order (or concurrently) without altering the principles of the present disclosure. Further, although each of the embodiments is described above as having certain features, any one or more of those features described with respect to any embodiment of the disclosure can be implemented in and/or combined with features of any of the other embodiments, even if that combination is not explicitly described. In other words, the described embodiments are not mutually exclusive, and permutations of one or more embodiments with one another remain within the scope of this disclosure.

Spatial and functional relationships between elements (for example, between modules, circuit elements, semiconductor layers, etc.) are described using various terms, including “connected,” “engaged,” “coupled,” “adjacent,” “next to,” “on top of,” “above,” “below,” and “disposed.” Unless explicitly described as being “direct,” when a relationship between first and second elements is described in the above disclosure, that relationship can be a direct relationship where no other intervening elements are present between the first and second elements, but can also be an indirect relationship where one or more intervening elements are present (either spatially or functionally) between the first and second elements. As used herein, the phrase at least one of A, B, and C should be construed to mean a logical (A OR B OR C), using a non-exclusive logical OR, and should not be construed to mean “at least one of A, at least one of B, and at least one of C.”

In the figures, the direction of an arrow, as indicated by the arrowhead, generally demonstrates the flow of information (such as data or instructions) that is of interest to the illustration. For example, when element A and element B exchange a variety of information but information transmitted from element A to element B is relevant to the illustration, the arrow may point from element A to element B. This unidirectional arrow does not imply that no other information is transmitted from element B to element A. Further, for information sent from element A to element B, element B may send requests for, or receipt acknowledgements of, the information to element A.

In this application, including the definitions below, the term “module” or the term “controller” may be replaced with the term “circuit.” The term “module” may refer to, be part of, or include: an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor circuit (shared, dedicated, or group) that executes code; a memory circuit (shared, dedicated, or group) that stores code executed by the processor circuit; other suitable hardware components that provide the described functionality; or a combination of some or all of the above, such as in a system-on-chip.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

The term code, as used above, may include software, firmware, and/or microcode, and may refer to programs, routines, functions, classes, data structures, and/or objects. The term shared processor circuit encompasses a single processor circuit that executes some or all code from multiple modules. The term group processor circuit encompasses a processor circuit that, in combination with additional processor circuits, executes some or all code from one or more modules. References to multiple processor circuits encompass multiple processor circuits on discrete dies, multiple processor circuits on a single die, multiple cores of a single processor circuit, multiple threads of a single processor circuit, or a combination of the above. The term shared memory circuit encompasses a single memory circuit that stores some or all code from multiple modules. The term group memory circuit encompasses a memory circuit that, in combination with additional memories, stores some or all code from one or more modules.

The term memory circuit is a subset of the term computer-readable medium. The term computer-readable medium, as used herein, does not encompass transitory electrical or electromagnetic signals propagating through a medium (such as on a carrier wave); the term computer-readable medium may therefore be considered tangible and non-transitory. Non-limiting examples of a non-transitory, tangible computer-readable medium are nonvolatile memory circuits (such as a flash memory circuit, an erasable programmable read-only memory circuit, or a mask read-only memory circuit), volatile memory circuits (such as a static random access memory circuit or a dynamic random access memory circuit), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a general purpose computer to execute one or more particular functions embodied in computer programs. The functional blocks, flowchart components, and other elements described above serve as software specifications, which can be translated into the computer programs by the routine work of a skilled technician or programmer.

The computer programs include processor-executable instructions that are stored on at least one non-transitory, tangible computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation) (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Swift, Haskell, Go, SQL, R, Lisp, Java®, Fortran, Perl, Pascal, Curl, OCaml, Javascript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

None of the elements recited in the claims are intended to be a means-plus-function element within the meaning of 35 U.S.C. § 112(f) unless an element is expressly recited using the phrase “means for,” or in the case of a method claim using the phrases “operation for” or “step for.”

What is claimed is:

1. A system comprising:
a reservoir located in a vehicle, the reservoir configured to store a substance for eliminating at least one of germs and odors from an interior of the vehicle;
a dispenser fluidly coupled to the reservoir, the dispenser configured to dispense the substance into the interior of the vehicle;
a pump configured to pump the substance from the reservoir to the dispenser; and
a controller configured to:
detect when an occupant of the vehicle exits the vehicle; and
in response to detecting the occupant of the vehicle exiting the vehicle, activate the pump to dispense the substance from the dispenser into the interior of the vehicle to eliminate at least one of the germs and the odors from the interior of the vehicle.

2. The system of claim 1 wherein the controller is configured to detect when the occupant of the vehicle exits the vehicle based on an operation of a door of the vehicle.

3. The system of claim 1 wherein the controller is configured to detect when the occupant of the vehicle exits the vehicle based on sensing a change of weight on a seat of the vehicle.

4. The system of claim 1 wherein the controller is configured to activate the pump after a predetermined amount of time has elapsed subsequent to the occupant exiting the vehicle.

5. The system of claim 1 wherein the controller is configured to activate the pump after a predetermined amount of time has elapsed subsequent to the vehicle being turned off.

6. The system of claim 1 wherein the reservoir and the pump are integrated as a single unit that is connected to the dispenser by a conduit.

7. The system of claim 1 wherein the reservoir, the pump, and the dispenser are integrated as a single unit.

8. The system of claim 1 further comprising conduits configured to connect the reservoir, the pump, and the dispenser.

9. The system of claim 1 further comprising:
a sensor configured to sense a level of the substance in the reservoir; and
an indicator configured to indicate the level of the substance in the reservoir.

10. The system of claim 1 further comprising:
a sensor configured to sense a level of the substance in the reservoir,
wherein the controller is configured to provide an indication of the level of the substance in the reservoir sensed by the sensor.

11. The system of claim 1 wherein at least one of the reservoir and the pump communicates wirelessly with the controller.

12. The system of claim 1 wherein the reservoir and the pump are integrated as a single unit that is connected to the dispenser by a conduit and that communicates wirelessly with the controller.

13. The system of claim 1 wherein the reservoir, the pump, and the dispenser are integrated as a single unit that communicates wirelessly with the controller.

14. A method comprising:
storing, in a reservoir located in a vehicle, a substance for eliminating at least one of germs and odors from an interior of the vehicle;
detecting when an occupant of the vehicle exits the vehicle; and
in response to detecting the occupant of the vehicle exiting the vehicle:
activating a pump to dispense the substance from a dispenser into the interior of the vehicle;
pumping the substance from the reservoir to the dispenser; and
dispensing the substance from the dispenser into the interior of the vehicle to eliminate at least one of the germs and the odors from the interior of the vehicle.

15. The method of claim 14 further comprising detecting when the occupant of the vehicle exits the vehicle based on an operation of a door of the vehicle.

16. The method of claim 14 further comprising detecting when the occupant of the vehicle exits the vehicle based on sensing a change of weight on a seat of the vehicle.

17. The method of claim 14 further comprising activating the pump after a predetermined amount of time has elapsed subsequent to the occupant exiting the vehicle.

18. The method of claim 14 further comprising activating the pump after a predetermined amount of time has elapsed subsequent to the vehicle being turned off.

19. The method of claim 14 further comprising:
sensing a level of the substance in the reservoir; and
indicating the level of the substance in the reservoir.

20. The method of claim 14 further comprising wirelessly communicating with at least the pump.
